(19) United States
(12) Patent Application Publication
(10) Pub. No.: US 2011/0318421 A1
(43) Pub. Date: Dec. 29, 2011

(54) SURFACE FUNCTIONALIZED CERAMIC NANOPARTICLES

(75) Inventor: Edward Parsonage, Saint Paul, MN (US)

(73) Assignee: Edward E. Parsonage, St. Paul, MN (US)

(21) Appl. No.: 13/166,756

(22) Filed: Aug. 8, 2011

Related U.S. Application Data
(60) Provisional application No. 61/398,479, filed on Jun. 24, 2010.
(60) Provisional application No. 61/398,480, filed on Jun. 24, 2010.

(51) Int. Cl. A61K 9/14; A61K 38/18; A61K 39/395; A61P 31/00; A61P 29/00; A61P 35/00; A61P 37/06; A61P 17/02; A61P 9/08; A61P 39/06; C08G 63/91; C08F 26/10; C08F 8/40; B32B 1/02; B32B 1/08; B32B 5/02; C07K 17/14; B82Y 30/00

(52) U.S. Cl. 424/490; 530/356; 530/410; 530/391.1; 514/8.8; 424/178.1; 525/450; 525/54.3; 428/221; 428/34.4; 977/773

(57) ABSTRACT

The present disclosure is directed to surface functionalized ceramic nanoparticles. The method for producing the surface functionalized ceramic nanoparticles generally includes at least four distinct steps: 1) synthesis of an amphiphilic surfactant having the desired surface functionality, 2) formation of mixed solvent microstructured solution with the surfactant, 3) synthesis of the desired ceramic within the microstructured solution, and 4) chemical attachment of the surfactant to the ceramic nanoparticle. The composition of the surface functionalized nanoparticle comprises a lipophilic component, a hydrophilic component, a chelating agent and a ceramic forming component.

### CROSS-REFERENCE TO RELATED APPLICATIONS

This application claims the benefit of U.S. Provisional Patent Application Ser. No. 61/398,479 and U.S. Provisional Patent Application Ser. No. 61/398,480, both applications filed Jun. 24, 2010 which applications are hereby incorporated by reference in their entirety.

### FIELD

The disclosure relates to surface functionalized ceramic nanoparticles. The disclosure specifically relates to surface functionalized nanoparticles comprising an amphiphilic surfactant and a chelating agent attached to a ceramic nanoparticle.

### BACKGROUND

Biodegradable materials are commonly used in areas ranging from medical devices to food packaging. For example, bioresorbable orthopaedic reinforcing members such as plates, pins and screws are of continuing interest because they have the potential to provide the mechanical functions required of trauma fracture fixation elements while at the same time eliminating various long-term effects associated with metallic implants.

However current polymeric biodegradable orthopaedic devices generally suffer from both insufficient acute load bearing capability and a delayed clinical healing response. The strength of current polymeric orthopaedic devices is limited simply by the narrow range of physical properties for the few biodegradable plastics approved for clinical use. Furthermore, without being bound by theory, it is believed that the non-biomimetic nature of today's materials exacerbates the foreign body inflammatory responses, reduces osteo-conductivity, and ultimately delays bone growth into the region of implant.

An advantage of the disclosure herein is that the surface functionalization of the ceramic nanoparticle is incorporated into the nanoparticle formation process, thus reducing any additional processing steps to achieve the desired surface chemistry.

Another advantage of the disclosure herein is that the surface functionalized ceramic nanoparticles have superior compatibility with other matrix materials resulting in improved properties such as, for example, physical strength.

Other non-medical applications for biodegradable plastics such as packaging films and injected molded parts suffer from similar limitations in available physical properties versus the broad range of biostable thermoplastic resins available today. This disclosure relates to a composition and method for producing a surface functionalized ceramic nanoparticle that may act as synergistic additives to advance a number of applications available to renewably derived bioplastics. Historical approaches to synthesizing such additives have fallen short, primarily because the additive particles were not specifically designed to be compatible with the polymer matrix, or the process for making the particles required too many steps to be feasible as a manufacturing process.

### SUMMARY

The present disclosure is directed to surface functionalized ceramic nanoparticles. The method for producing surface functionalized ceramic nanoparticles generally includes at least four distinct steps: 1) synthesis of an amphiphilic surfactant having the desired surface functionality for utility within the targeted application, 2) formation of a multiphase mixed solvent microstructured solution with the surfactant, 3) synthesis of the desired ceramic within the microstructured solution, and 4) chemical attachment of the surfactant to the ceramic nanoparticle.

An embodiment of the composition of the functionalized ceramic nanoparticle includes compositions having the formula:

(S_{ABX})—C

wherein S describes a surfactant comprising the components of A, B, and X; and C is a ceramic.

In another embodiment a unique functionalized ceramic nanoparticle composition is formed from an AB molecular surfactant having oil affinitive component A and water affinitive component B. The water affinitive component B also contains a ceramic chelating agent X, resulting in the final surfactant structure ABX. An oil/water microemulsion is then formed using the surfactant ABX, with the surfactant concentrating at the oil/water interface and the A component associated predominately with the oil phase and the BX component with the water phase. Subsequent to microemulsion formation, a ceramic forming composition is then added to the microemulsion, resulting in both precipitation of ceramic nanoparticles within the aqueous phase and chemical attachment through chelating agent X. The attached surfactant ABX forms the surface functionality of the final ceramic nanoparticle.

In another embodiment, the surface functionality imparted by the chemically attached surfactant comprises a biodegradable polymer, and the ceramic nanoparticle comprises a bioresorbable bioceramic, such as bone biomimetic hydroxyapatite or carbonate apatite.

In another embodiment the surface functionalization may include a biologic such as an antibody, and the ceramic core may also contain therapeutic agents. Such a construction could be particularly useful for drug delivery applications.

### BRIEF DESCRIPTION OF THE DRAWINGS

FIG. 1 is a schematic perspective view of one configuration of the synthesized surfactant ABX having lipophilic component A, hydrophilic component B, and chelating agent X.

FIG. 2 is a schematic perspective view of the microemulsion formation of water phase C, oil phase D, ceramic particle precipitation E/F, and chemical attachment of the surfactant.

FIG. 3 is a schematic perspective view of one configuration of the surface functionalized nanoparticle.

FIG. 4 is a transmission electron microscope image of the nanoparticle powder synthesized using 33% v/v water.

FIG. 5 is a transmission electron microscope image of the nanoparticle powder synthesized using 5% v/v water.

### DETAILED DESCRIPTION OF THE EMBODIMENTS

As noted above, the embodiments are directed to surface functionalized ceramic nanoparticles. The method for forming surface functionalized ceramic nanoparticles generally includes the steps of 1) synthesis of a surfactant possessing at least one specific agent capable of chemical attachment or chelating to the ceramic particle, 2) formation of a microstructured solution with the surfactant using a combination of incompatible solvents having different polarities, 3) addition of ceramic pre-cursor compositions resulting in ceramic nanoparticle precipitation within the microemulsion, and 4) chemical attachment of the chelating functionality to the ceramic nanoparticle surface.

In accordance with an embodiment of the method, an amphiphilic AB molecular surfactant is synthesized having oil affinitive component A and water affinitive component B. Synthesis of the surfactant further includes incorporation of a chelating agent X capable of chemical attachment with the ceramic, resulting in the final surfactant structure ABX. A microstructured solution is then formed via an oil water microemulsion using the surfactant ABX, with the surfactant self-assembling at the oil/water interface and the A component associated predominately within the oil phase and the BX component within the water phase. Subsequent to microemulsion formation, a ceramic forming composition is then added to microemulsion, resulting in both precipitation of ceramic nanoparticles within the aqueous phase and attachment to surfactant ABX. The attached surfactant ABX thus forms the surface functionality of the final ceramic nanoparticle as, for example, a coating over the ceramic nanoparticle. Such a coating is of uniform thickness over the exterior of the ceramic nanoparticle. The surface functionality provided by surfactant ABX is completely dispersed and available over the surface of the nanoparticle.

An embodiment of the composition of the functionalized ceramic nanoparticle includes compositions having the formula:

(S_{ABX})—C

wherein S describes a surfactant comprising the components of A, B, and X; and C is a ceramic.

The lipophilic component A and the hydrophilic component B may be selected from groups comprising a biostable polymer, a biodegradable polymer, a biopolymer, or a biologic. The surfactant may also include any number of additional hydrophilic, lipophilic and chelating components in the composition.

The chelating agent X may be located at the distal end of the hydrophilic component B. Alternately, chelating agent X may be located along the backbone of the A component or B component, or at the terminal end of the A component. Furthermore, the chelating agent X may also comprise the hydrophilic component of the surfactant.

Presence of the chelating agent X results in attachment of the surfactant to the ceramic nanoparticle during the precipitation process. In general, the mechanism of attachment may arise from any one or combination of interactions including covalent, electrostatic, hydrogen bonding, Van der Waals interaction and/or mechanical interlocking.

In another embodiment application of an ultrasonic agitation may be applied to the microemulsion to facilitate formation of the structured solution and mixing of the ceramic precursors into the aqueous phase.

In another embodiment application of a microwave energy source to the microemulsion reaction medium may be used. Without being bound by theory, in addition to controlling the temperature of the microemulsion, it is also believed that the oscillating electromagnetic microwave field acts to direct nanoparticle synthesis through molecular alignment of the polar precursor components.

Further embodiments include the incorporation of isolation and purification process steps including precipitation, washing, grinding and filtering.

In an embodiment the surface functionalized nanoparticles may be combined with a bulk matrix material to create a device or article. The matrix material may be a biodegradable material such as a biodegradable plastic or bioresorbable ceramic. Alternately, the matrix material may be a biostable material.

In one example, the surface functionalized nanoparticles are combined with a biodegradable plastic to form a bioresorbable orthopaedic reinforcing members such as plates, pins, intramedullary rods and screws. Other embodiments may include cements used for bone and dental applications.

In another example, the surface functionalized nanoparticles are incorporated into a semi-crystalline biodegradable plastic as nucleating agents to promote crystallization. In a more preferred embodiment, the surface functionalization comprises a stereocomplex of a biodegradable plastic. Additionally, the surface functionalized nanoparticle may be added to a biodegradable plastic to act as impact modifying agents.

Other embodiments for the surface functionalized nanoparticles may include carriers for drug delivery and fertilizers.

The surfactant component contains an oil affinitive lipophilic component A, water affinitive hydrophilic component B, and chelating agent X.

As defined herein, a component is lipophilic if, when placed in an oil/water suspension, preferentially concentrates in the oil phase. Similarly, a component is hydrophilic if, when placed in an oil/water suspension, preferentially concentrates in the aqueous phase.

The surfactant is comprised of at least one lipophilic component, at least one hydrophilic component, and at least one chelating agent such that the surfactant is able to aggregate at the oil/water interface to form a microstructured solution. Materials for forming the surfactant components comprise biostable and biodegradable polymeric materials, biopolymers and biologics.

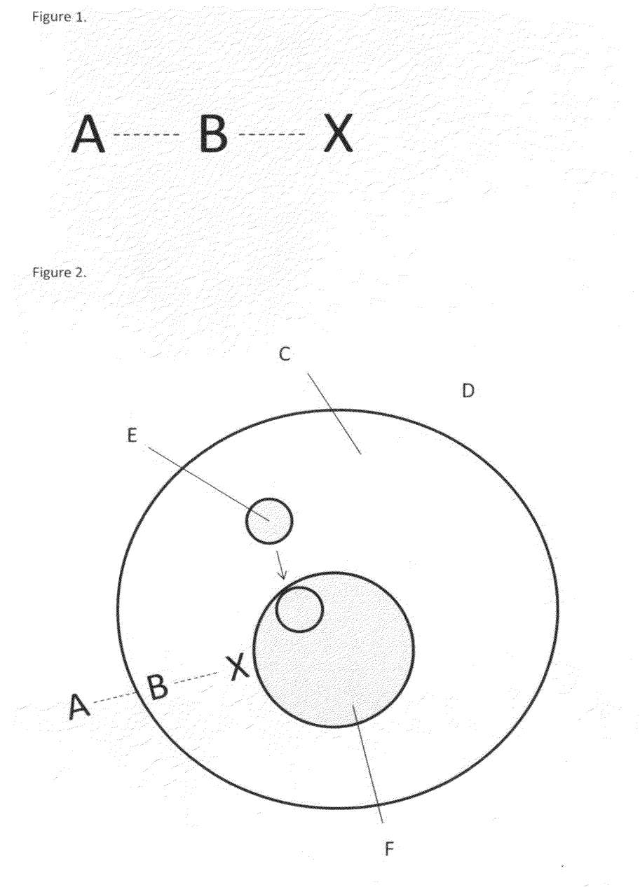

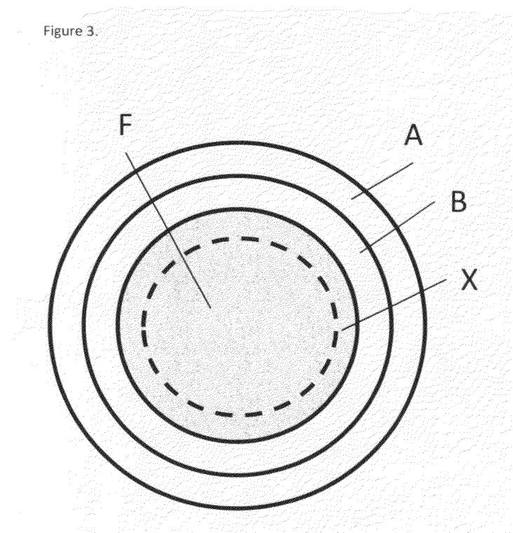

An oil/water microemulsion is formed using the surfactant ABX, with the surfactant concentrating at the oil/water interface and the A component associated predominately with the oil phase and the BX component with the water phase (FIG. 1). A unique functionalized ceramic nanoparticle composition is formed from an AB molecular surfactant having oil affinitive component A and water affinitive component B. The water affinitive component B also contains a ceramic chelating agent X, resulting in the final surfactant structure ABX. Subsequent to microemulsion formation, a ceramic forming composition is then added to the microemulsion, resulting in both precipitation of ceramic nanoparticles within the aqueous phase and chemical attachment through chelating agent X. (FIG. 2) The attached surfactant ABX forms the surface functionality of the final ceramic nanoparticle. (FIG. 3).

Chemical coupling of the surfactant components may be achieved by any number of means known in the chemical art. For example, end groups of the respective components may be combined together through commonly known condensation reactions such as, for example, esterification, amidation, urethane and urea formation. Alternately, a leaving agent such as a diazonium salts, tosylates, triflates and halides, may be used to facilitate or catalyze the coupling reactions.

As defined herein, “polymers” are molecules containing multiple copies (e.g., 3 or more copies) of one or more constitutional units, commonly referred to as monomers. As used defined, “homopolymers” are polymers that contain multiple copies of a single constitutional unit. “Copolymers” are polymers that contain multiple copies of at least two dissimilar constitutional units, examples of which include random, statistical, gradient, periodic (e.g., alternating) and block copolymers. As used herein, a polymer is “biodegradable” if it undergoes bond cleavage along the polymer backbone either in vivo, or in the environment under conditions such as in a landfill, composting, or recycling process, regardless of the mechanism of bond cleavage (e.g., enzymatic breakdown, hydrolysis, oxidation, etc.). Alternately, a biostable polymer does not undergo biodegradation over the intended lifetime of the device or product.

Biopolymers are defined as polymers which are synthesized naturally such as, for example, peptides, proteins, phospholipids, starches, collagen and fibrin. Similarly, biologics comprise biopolymers which have specific biological activity such as, for example, antibodies, antigens, cellular receptors, lipoproteins, enzymes, integrins, fibronectins, kinases, growth factors, and interference RNA.

Examples of biodegradable polymers may be selected from the following: (a) polyester homopolymers and copolymers such as polyglycolide (PGA) (also referred to as polyglycolic acid), polylactide (PLA) (also referred to as polylactic acid) including poly-L-lactide, poly-D-lactide and poly-D,L-lactide, poly(beta-hydroxybutyrate), polygluconate including poly-D-gluconate, poly-L-gluconate, poly-D,L-gluconate, poly(epsilon-caprolactone), poly(delta-valerolactone), poly(p-dioxanone), poly(lactide-co-glycolide) (PLGA), poly(lactide-co-delta-valerolactone), poly(lactide-co-epsilon-caprolactone), poly(lactide-co-beta-malic acid), poly(beta-hydroxybutyrate-co-beta-hydroxyvalerate), poly[1,3-bis(p-carboxyphenoxy)propane-co-sebacic acid], and poly(sebacic acid-co-fumaric acid), among others, (b) polycarbonate homopolymers and copolymers such as poly(trimethylene carbonate), poly(lactide-co-trimethylene carbonate) and poly(glycolide-co-trimethylene carbonate), among others, (c) poly(ortho ester) homopolymers and copolymers such as those synthesized by copolymerization of various diketene acetals and diols, among others, (d) polyanhydride homopolymers and copolymers such as poly(adipic anhydride), poly(suberic anhydride), poly(sebacic anhydride), poly(dodecanedioic anhydride), poly(maleic anhydride), poly[1,3-bis(p-carboxyphenoxy)methane anhydride], and poly[alpha,omega-bis(p-carboxyphenoxy)alkane anhydrides] such as poly[1,3-bis(p-carboxyphenoxy)propane anhydride] and poly[1,3-bis(p-carboxyphenoxy)hexane anhydride], among others, (e) polyphosphazenes such as aminated and alkoxy substituted polyphosphazenes, and (f) amino-acid-based polymers including tyrosine-based polymers such as tyrosine-based polyarylates (e.g., copolymers of a diphenol and a diacid linked by ester bonds, with diphenols selected, for instance, from ethyl, butyl, hexyl, octyl and bezyl esters of desaminotyrosyl-tyrosine and diacids selected, for instance, from succinic, glutaric, adipic, suberic and sebacic acid), tyrosine-based polycarbonates (e.g., copolymers formed by the condensation polymerization of phosgene and a diphenol selected, for instance, from ethyl, butyl, hexyl, octyl and benzyl esters of desaminotyrosyl-tyrosine), tyrosine-based iminocarbonates, and tyrosine-, leucine- and lysine-based polyester-amides; specific examples of tyrosine-based polymers further include polymers that are comprised of a combination of desaminotyrosyl tyrosine hexyl ester, desaminotyrosyl tyrosine, and various di-acids, for example, succinic acid and adipic acid, among others.

In an embodiment, biodegradable polymers are polymers comprised of poly(lactic acid), poly(glycolic acid), poly(caprolactone), and various copolymers thereof are used. Useful molecular weights for the biodegradable polymers may range, for example, from oligomers comprised of several to tens of repeat units and having molecular weights from 500 g/mol to 10,000 g/mol to polymers having hundreds to thousands of repeat and molecular weights from 10,000 g/mol to over 1,000,000 g/mol. For further information on biodegradable polymers see, e.g., Handbook of Biodegradable Polymers, Abraham J. Domb, Joseph Kost, David M. Wiseman, Eds., CRC Press, 1997 herein incorporated by reference in its entirety.

Examples of suitable biostable polymers include, for example, saturated and unsaturated polyolefins such as polyethylene; polyacrylics; polyacrylates; polymethacrylates; polyamides; polyimides; polyurethanes; polyureas; polyvinyl aromatics such as polystyrene; polyisobutylene copolymers and isobutylene-styrene block copolymers such as styrene-isobutylene-styrene tert-block copolymers (SIBS); polyvinylpyrolidone; polyvinyl alcohols; copolymers of vinyl monomers such as ethylene vinyl acetate (EVA); polyvinyl ethers; polyesters including polyethylene terephthalate; polyacrylamides; polyethers such as polyethylene glycol, polytetrahydrofuran and polyether sulfone; polycarbonates; silicones such as siloxane polymers; cellulosic polymers such as cellulose acetate; and fluoropolymers such as polyvinylidene fluoride; and mixtures and copolymers of any of the foregoing.

In an embodiment biostable polymers include poly(ethylene glycol), poly(vinyl alcohol) and poly(ethylene imine).

Useful molecular weights for the biostable polymers may range, for example, from oligomers comprised of several to tens of repeat units and having molecular weights from 500 g/mol to 10,000 g/mol to polymers having hundreds to thousands of repeat units and molecular weights from 10,000 g/mol to over 1,000,000 g/mol.

Biopolymers and biologics comprise, for example, the general categories of peptides, proteins, fatty acids, triglycerides, complex carbohydrates, oligonucleotides and nucleic acid polymers. Examples of these general categories are extracellular matrix proteins including collagen and collagen sub-units such as type I collagen, fibrin and fibrinogen; biological structures known to promote cellular adhesion such as the RGD peptide sequence and fibronectin; growth factors such as bone morphogenic proteins (“BMP’); carbohydrates such as starch and cellulose; plant cellular components including lignin and cellulose; proteins and peptides; cellular adhesion entities including antibodies and antigens; enzymes; oligonucleotides and nucleic acids such as double or single stranded DNA (including naked and cDNA), RNA, antisense nucleic acids such as antisense DNA and RNA, small interfering RNA (siRNA), and ribozymes; and genes. Particularly preferred biopolymers include collagen, starches, cellulose and antibodies.

In an embodiment, lipophilic components of the surfactant would include, for example, biodegradable polymer such as poly(glycolic acid) (PGA), polycaprolactone (PCL), poly(lactic acid) (PLA), stereocomplexes of poly(lactic acid), poly(lactide-co-glycolide) (PLGA); biostable polymers including polyolefins such as polyethylene, polyacrylates and polymethacrylates such as poly(n-butyl acrylate), biopolymers including long chain fatty acids and triglycerides such as lecithin, collagens and fibrinogens. In an embodiment, hydrophilic components of the surfactant would include, for example, water soluble oligo-lactides, poly(ethylene oxide) (PEO), poly(ethylene glycol) (PEG), poly(vinyl alcohol) (PVA), poly(vinyl pyrolidone) (PVP), poly(ethylene amine) (PEA), poly(acrylic acid) (PAA), cholines such as N,N,N-(polyethylene glycol) ethanolammonium phosphate, water soluble peptides such as poly(alanine) and complex carbohydrates such as chitosan.

The chelating component X of the surfactant is defined as a chemical moiety capable of attachment to the surface or near surface region of the ceramic nanoparticle. The attachment of component X to the ceramic nanoparticle may arise from any number of molecular interactions ranging from predominately covalent bonding involving sharing of electron pairs, to predominately ionic bonding involving interaction of monopolar, dipolar and higher polarity structures, to mechanical interlocking of the surfactant within the near surface region of the ceramic nanoparticle.

In general, the chelating agent X may be attached anywhere along the surfactant. In an embodiment, the chelating agent is attached to the hydrophilic component B of the surfactant. In another embodiment, the chelating agent X comprises the end group of component B. In a further embodiment, the chelating agent itself may comprise the hydrophilic component B.

Examples of chelating agents comprising X may include, for example, dipolar compositions having a general structure YZ, where Y is an electronegative (anionic) component and Z is an electropositive (cationic) component. Examples of the electronegative component Y may include, for example, oxyanions such as carboxylates; sulfates; phosphates; nitrates; chlorates; silicates; chromates; molybdates; carbonates; and alkoxides. The electropositive cationic component Z may include, for example, acids; positively charged organometallic salts; protonated organic bases such as the ammonium cation NH4+, metallic cations such as Na+, K+, Li+, Mg++, and Ca++; and cationic onium structures such as trimethylammonium.

The chelating agent X comprises substantially non-ionic structures known to complex specifically with cationic components. Specifically, multidentate molecular ligands capable of occupying more than one space in the coordination sphere of the metal ion to form a cyclic structure. These cationic binding agents would include, for example, polyethers including ring shaped crown ethers such as 18-crown-6, polyamines such as ethylene diamine triacetic acid (EDTA), dithiols, thiocarbamates, and phosphates such as 1,2 ethylene diphosphonic acid.

In an embodiment chelating agent X comprises carboxylate acids and salts, phosphate acids and salts, polyethers and EDTA.

Optional components of the surfactant may comprise, for example, structures that have no particular affinity for oil versus water such as fluorinated compounds. Other excipients and additives may be included as well.

The microemulsion is defined as a microstructured solution formed by two or more immiscible solvents of different polarity, and a surfactant which stabilizes the emulsion by aggregating at the interfacial regions between the solvents. The hydrophilic component of the surfactant is predominately located within the more polar solvent phase, and the lipophilic component of the surfactant predominately located within the less polymer solvent phase. The microemulsion formed with the surfactant may comprise any number of meso-phases known to form in such systems depending on the surfactant hydrophilic-lipophilic balance and volumetric ratio of the solvent phases. Examples of such phases would include, but are not limited to, spherical micelles, cylinders, platelets, bicontinuous structures and larger liposomes. For further information on microemulsions see, e.g., On the Origins of Morphological Complexity in Block Copolymer Surfactants, Sumeet Jain and Frank S. Bates, Science 18 Apr. 2003: Vol. 300. no. 5618, pp. 460-464 herein incorporated by reference in its entirety.

In general, two or more immiscible solvents of differing polarity are required to form the microemulsion. Non-polar solvents are typically defined as having a dielectric constant of less than 15. Examples of polar solvents include water, alcohols such as isopropyl alcohol. Non-polar solvents include, for example, hexane, tetrachloroethylene, benzene, toluene, ethyl acetate, and diethyl ether for example. Polar aprotic solvents or those that do not include a hydrogen ion, include, for example, 1,4-dioxane, tetrahydrofuran, dichloromethane, acetone, acetonitrile, dimethylformamide, and dimethyl sulfoxide for example. Polar protic solvents or those having a hydrogen ion bound to an oxygen include, for example, water, alcohols such as isopropanol, ethanol, n-butanol and methanol, acetic acid, and formic acid. In an embodiment the solvents are water, tetrahydrofuran and ethyl acetate. In general, the volumetric ratio of solvents may partially determine the solution microstructure. The range of polar solvent may vary from 0.1 to 90%, 1% to 50%, or 10% to 30%.

In general, the ceramic precursors are comprised of ionic solutions that, when mixed in aqueous solutions, are known to form ceramic precipitants. For purposes herein, a ceramic is defined as a solid substance having melting points greater than 100° C. and comprised of metallic cationic components and oxygen containing anionic components. The metallic cationic components may comprise monovalent and polyvalent elements such as, for example, Li, K, Na, Mg, Ca, and Si. The oxygen anionic components include, for example, sulfates, phosphates, nitrates, chlorates; silicates, chromates, molybdates, and carbonates.

In an embodiment, ceramic precursors may include, for example, calcium chloride, calcium oxide, calcium hydroxide, calcium nitrate, magnesium hydroxide, magnesium chloride, potassium hydroxide, potassium chloride, phosphoric acid, carbonic acid, carbon dioxide, sulfuric acid, sodium phosphate, mono and di-basic sodium phosphate, mono and di-basic potassium phosphate, ammonium carbonate, ammonium phosphate, calcium acetate, and calcium citrate.

In another embodiment, ceramic precursors may be calcium chloride, calcium nitrate, ammonium phosphate and sodium phosphate.

Typical concentrations for surfactant precursors within the microemulsion may range from 1 to 1000 mM, more preferably 10-100 mM.

Other components may be included within the ceramic precursors, such as, for example additives required to control the solution pH. Examples of such pH controlling reagents would include acids such as hydrochloric acid, bases such as ammonium hydroxide, and buffers such as tris(hydroxymethyl)aminomethane (TRIS) or mixtures thereof. Concentration ranges for pH controlling agents typically range from 1 to 100 mM.

Other components may be incorporated within the ceramic nanoparticle precipitation process such as one or more therapeutic agents. The therapeutic agent may be any genetic, biological, bioactive, therapeutic material or agent which may provide a desired effect. Suitable therapeutic agents include pharmaceuticals, genetic materials, and biological materials. For example, in some embodiments, the therapeutic agent may include a drug which may be used in the treatment of infection. Some suitable therapeutic agents which may be loaded in the surface functionalized ceramic nanoparticle include, for example, antibiotics, antimicrobials, anti-inflammatories, growth factors, anti-proliferatives, anti-neoplastics, antioxidants, endothelial cell growth factors, thrombin inhibitors, immunosuppressants, anti-platelet aggregation agents, collagen synthesis inhibitors, therapeutic antibodies, nitric oxide donors, antisense oligonucleotides, wound healing agents, therapeutic gene transfer constructs, peptides, proteins, extracellular matrix components, vasodialators, thrombolytics, anti-metabolites, growth factor agonists, antimitotics, steroidal and non-steroidal anti-inflammatory agents, angiotensin converting enzyme (ACE) inhibitors, free radical scavengers, and anticancer chemotherapeutic agents.

The term “therapeutic agent” encompasses pharmaceuticals, genetic materials, and biological materials. Examples of suitable therapeutic agents include heparin, heparin derivatives, urokinase, dextrophenylalanine proline arginine chloromethylketone (PPack), enoxaprin, angiopeptin, hirudin, acetylsalicylic acid, tacrolimus, everolimus, rapamycin (sirolimus), amlodipine, doxazosin, glucocorticoids, betamethasone, dexamethasone, prednisolone, corticosterone, budesonide, sulfasalazine, rosiglitazone, mycophenolic acid, mesalamine, paclitaxel, 5-fluorouracil, cisplatin, vinblastine, vincristine, epothilones, methotrexate, azathioprine, adriamycin, mutamycin, endostatin, angiostatin, thymidine kinase inhibitors, cladribine, lidocaine, bupivacaine, ropivacaine, D-Phe-Pro-Arg chloromethyl ketone, platelet receptor antagonists, anti thrombin antibodies, anti platelet receptor antibodies, aspirin, dipyridamole, protamine, hirudin, prostaglandin inhibitors, platelet inhibitors, trapidil, liprostin, tick antiplatelet peptides, 5-azacytidine, vascular endothelial growth factors, growth factor receptors, transcriptional activators, translational promoters, antiproliferative agents, growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin, cholesterol lowering agents, vasodilating agents, agents which interfere with endogenous vasoactive mechanisms, antioxidants, probucol, antibiotic agents, penicillin, cefoxitin, oxacillin, tobranycin, angiogenic substances, fibroblast growth factors, estrogen, estradiol (E2), estriol (E3), 17-beta estradiol, digoxin, beta blockers, captopril, enalopril, statins, steroids, vitamins, taxol, paclitaxel, 2′-succinyl-taxol, 2′-succinyl-taxol triethanolamine, 2′-glutaryl-taxol, 2′-glutaryl-taxol triethanolamine salt, 2′-O-ester with N-(dimethylamino ethyl)glutamine, 2′-O-ester with N-(dimethylaminoethyl)glutamide hydrochloride salt, nitroglycerin, nitrous oxides, nitric oxides, antibiotics, aspirins, digitalis, estrogen, estradiol and glycosides. In one embodiment, the therapeutic agent is an antibiotic such as erythromycin, amphotericin, rapamycin, adriamycin, etc.

The term “genetic materials” means DNA or RNA, including, without limitation, DNA/RNA encoding of a useful protein as stated below and intended to be inserted into a human body including viral vectors and non-viral vectors.

The term “biological materials” include, for example, cells, yeasts, bacteria, proteins, peptides, cytokines and hormones. Examples for peptides and proteins include vascular endothelial growth factor (VEGF), transforming growth factor (TGF), fibroblast growth factor (FGF), epidermal growth factor (EGF), cartilage growth factor (CGF), nerve growth factor (NGF), keratinocyte growth factor (KGF), skeletal growth factor (SGF), osteoblast-derived growth factor (BDGF), hepatocyte growth factor (HGF), insulin-like growth factor (IGF), cytokine growth factors (CGF), platelet-derived growth factor (PDGF), hypoxia inducible factor-1 (HIF-1), stem cell derived factor (SDF), stem cell factor (SCF), endothelial cell growth supplement (ECGS), granulocyte macrophage colony stimulating factor (GM-CSF), growth differentiation factor (GDF), integrin modulating factor (IMF), calmodulin (CaM), thymidine kinase (TK), tumor necrosis factor (TNF), growth hormone (GH), bone morphogenic protein (BMP) (e.g., BMP-2, BMP-3, BMP-4, BMP-5, BMP-6 (Vgr-1), BMP-7 (PO-1), BMP-8, BMP-9, BMP-10, BMP-11, BMP-12, BMP-14, BMP-15, BMP-16, etc.), matrix metalloproteinase (MMP), tissue inhibitor of matrix metalloproteinase (TIMP), cytokines, interleukin (e.g., IL-1, IL-2, IL-3, IL-4, IL-5, IL-6, IL-7, IL-8, IL-9, IL-10, IL-11, IL-12, IL-15, etc.), lymphokines, interferon, integrin, collagen (all types), elastin, fibrillins, fibronectin, vitronectin, laminin, glycosaminoglycans, proteoglycans, transferrin, cytotactin, cell binding domains (e.g., RGD), and tenascin. Useful BMP's are BMP-2, BMP-3, BMP-4, BMP-5, BMP-6, BMP-7. These dimeric proteins may be provided as homodimers, heterodimers, or combinations thereof, alone or together with other molecules. Cells may be of human origin (autologous or allogeneic) or from an animal source (xenogeneic), genetically engineered, if desired, to deliver proteins of interest at the transplant site. The delivery media may be formulated as needed to maintain cell function and viability. Cells include progenitor cells (e.g., endothelial progenitor cells), stem cells (e.g., mesenchymal, hematopoietic, neuronal), stromal cells, parenchymal cells, undifferentiated cells, fibroblasts, macrophage, and satellite cells.

In other embodiments, the therapeutic agents for use in the medical devices of the present disclosure may be synthesized by methods well known to one skilled in the art. Alternatively, the therapeutic agents may be purchased from chemical and pharmaceutical companies.

Therapeutic agents may be used singly in combination or in mixtures. A wide range of therapeutic agent loadings may be used in conjunction with the devices of the present disclosure, with the pharmaceutically effective amount being readily determined by those of ordinary skill in the art and ultimately depending, for example, upon the condition to be treated, the nature of the agent itself, and the tissue into which the dosage form is introduced.

Mechanisms of drug delivery may comprise any of those known in the art including, for example, ingestible solutions and tablets, systemic injection, aerosol inhalers, nasal sprays, transdermal delivery, ocular delivery, and controlled release implants.

The ceramic precursors may be added to microemulsion in any sequence or manner. For example, the ceramic precursors may be added simultaneously or separately. Furthermore, the ceramic precursors may be added at once, or drop wise during the ceramic nanoparticle formation process. A subset of the ceramic precursors may be added prior to microemulsion formation. Surface functionalized ceramic nanoparticles formed have sizes of 1 to 1000 nanometers, more preferably 10 to 100 nanometers.

Additional process steps may be incorporated to facilitate the nanoparticle synthesis. Ultrasonic mixing may be applied to both create the microemulsion and facilitate mixing of the ceramic precursors. For example, the reaction vessel may be submerged into an ultrasonic mixing apparatus. Alternately, an ultrasonic horn may be attached to the side of the reaction vessel. Similarly, an electromagnetic energy source such as RF, IR or microwave, may be applied to control the reaction temperature and facilitate nanoparticle formation. Temperatures ranges would include 0° C. to 100° C., more preferable 25° C. to 50° C. Additional steps such as precipitation into non-solvents, purification and grinding may be applied to produce the final product.

The surface functionalized ceramic nanoparticles may be combined with other materials to produce various devices or articles. Examples of such articles may include, for example, composites of the surface functionalized nanoparticles with other bioresorbable matrix materials for medical devices such as orthopaedic reinforcing members; combination with other ceramic precursors to be used as bone and dental cements; combination with other materials to create biodegradable consumer disposable and durable goods such as food packaging and structural automotive elements. Device and article components in accordance with the present disclosure may be bioabsorbed by a subject upon implantation or insertion of the component into the subject. “Bioabsorption” or “bioresorption” of a polymer-containing medical device component is defined herein to be a result of polymer biodegradation (as well as other in vivo disintegration processes such as dissolution, etc.) and is characterized by a substantial loss in vivo over time (e.g., the period that the component is designed to reside in a patient) of the original polymer mass of the device or component. For example, losses may range from 50% to 75% to 90% to 95% to 97% to 99% or more of the original polymer mass of the device component. Bioabsorption times may vary widely, with typical bioabsorption times ranging from days to months to years, depending on the application.

The devices may be further combined with other components to make multilayer constructions such as films and tapes.

Devices may be fabricated, for example, by any number of means, including extrusion, melt spinning, injection molding, reaction injection molding, hot pressing, rapid prototyping, and solution casting.

### Example 1

In this example, 2 g of DL poly (lactic acid) (Lactel Absorbable Polymers, inherent viscosity 0.5 dL/g) was dissolved in 50 mL tetrahydrofuran (Sigma Aldrich >99% purity). To this was added 2 mL 0.037% aqueous hydrocholic acid, fluxed at 50 C for 2 hours, then neutralized with 0.2 mL 0.37% ammonium hydroxide. The sample was subsequently dried over molecular sieve (Sigma Aldrich 3 Å 4/8 mesh) for 24 hours. To this solution was added 0.3 g triethyl amine (Sigma Aldrich), 0.3 g tosyl chloride (Sigma Aldrich reagent grade >98%), and fluxed at 50 C for 2 hours. To this was added 0.8 g of an amino-carboxylate terminated polyethylene glycol (Sigma Aldrich O-(2-Aminoethyl)-O′-(2-carboxyethyl) polyethylene glycol 3,000 hydrochloride) and fluxed at 50 C for 24 hours. Subsequent to air drying, 0.33 g of the above polymer was dissolved into 80 cc ethyl acetate (Sigma Aldrich >99%) (solution S1). Two additional solutions were prepared: 0.5 g calcium chloride (purity >93%) in 10 cc deionized water (purity >99%) (solution S2), and 0.45 g trisodium phosphate (purity >96%) in 10 cc deionized water (solution S3) (all from Sigma Aldrich).

Surface functionalized nanoparticle synthesis was carried out by first immersing 40 cc of solution S1 in an ultrasonic water bath (VWR) at a temperature of 37 C. To this was then added 10 cc of solution S2. Next 10 cc of solution S3 was added drop-wise using a pipette to a final aqueous phase volumetric composition of 33%. Subsequent to final addition of solution S3, the glass reaction vessel was transferred to a microwave heater (Samsung), the power of which was cycled on/off to maintain an average temperature of 50 C for a period of 1 hour.

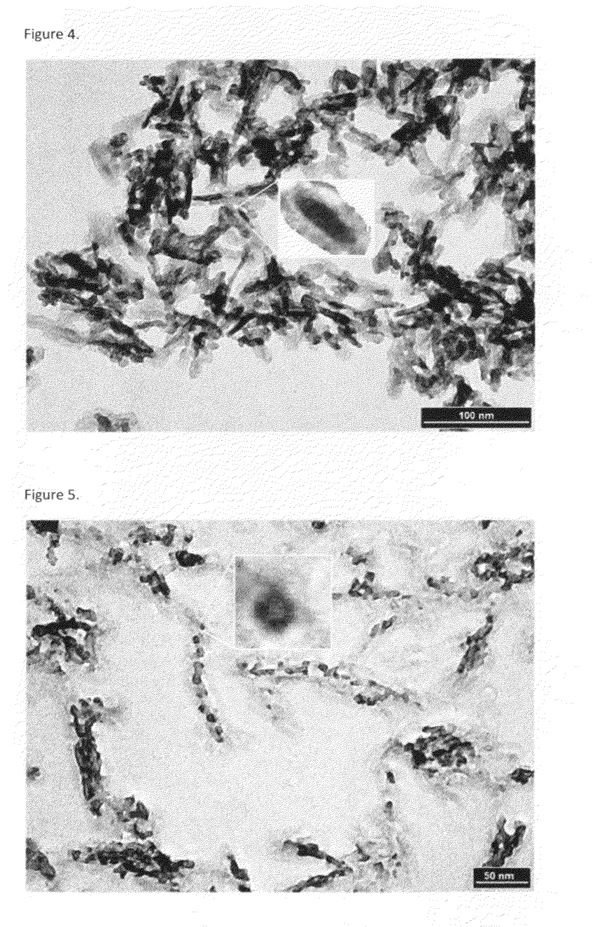

The reacted sample was then precipitated drop-wise into 160 mL isopropyl alcohol (Sigma Aldrich) and dried at 37 C for 2 hours. The resulting precipitate was then characterized by dispersing a sample of the powder in deionized water onto a transmission electron microscope (TEM) grid. FIG. 4 shows the subsequent TEM image indicating the nanoparticle structure.

### Example 2

In Example 2, the powder samples was prepared in samples, with the exception that 1 cc of solution S1 and 1 cc of solution S2 were added in the same manner to 40 cc solution S2 for a total aqueous component composition of 5%. FIG. 5 shows the subsequent TEM image indicating the nanoparticle structure.

Although various embodiments are specifically illustrated and described herein, it will be appreciated that modifications and variations of the present disclosure are covered by the above teachings and are within the purview of the appended claims without departing from the spirit and intended scope of the disclosure.

### Example 3

In Example 3, a composite sample comprising 33% by volume of the particles in Example 1 in a matrix of a poly (lactic acid-co-glycolic acid) copolymer [RESOMER® RG 503 Poly(D,L-lactide-co-glycolide) 50:50, Boehringer Ingelheim] was fabricated by mixing 20% by weight polymer and 10% by weight powder into an ethyl acetate solution, then casting into a uniform film of approximate thickness of 10 mils. The storage modulus of an approximately 5 mm×10 mm strip of the composite sample was then measured on a TA Instruments DMA Q800 Dynamic Mechanical Analyzer. The measured (T=20° C.) storage modulus was 2423 MPa.

### Comparative Example 4

In this Example, a pure polymer (no powder) film sample was prepared and tested as in Example 3, with the a poly (lactic acid-co-glycolic acid) polymer [RESOMER® RG 503 Poly(D,L-lactide-co-glycolide) 50:50, Boehringer Ingelheim] added at 30% by weight into the ethyl acetate solution. The measured storage (T=20° C.) modulus for this polymer only film was 1115 MPa.

### Comparative Example 5

In this Example, composite sample was prepared and tested as in Example 3, with the exception that the powder sample comprised a commercially available, non-surface functionalized hydroxyl apatite [Sigma Aldrich product no. 693863 nanopowder, <200 nm particle size]. The measured storage (T=20° C.) modulus for this non-surface functionalized powder was 1123 MPa.

The greater than factor of two increase in modulus for the sample prepared in Example 3 versus both no-filler (Example 4) and non-surface functionalized filler (Example 5) demonstrates the improvement in physical properties attained using the method of this disclosure.

### Example 6

In this Example, a powder is prepared as in Example 1, except the surfactant possesses a lipophilic component comprised of a poly(lactic acid-co-glycolic acid) copolymer, the hydrophilic component is a polyethylene glycol, the chelating agent is a carbonate, and the ceramic is a carbonate apatite.

### Example 7

In this Example, a powder is prepared as in Example 1, except the surfactant possesses a lipophilic component comprised of poly(caprolactone), the hydrophilic component is a polyethylene glycol, the chelating agent is a phosphate, and the ceramic is tricalcium phosphate.

### Example 8

In this Example, a powder is prepared as in Example 1, except the surfactant possesses a lipophilic component comprised of cellulose, the hydrophilic component is poly(vinyl pyrolidone), the chelating agent is a sulfate, and the ceramic is a calcium sulfate.

### Example 9

In this Example, a powder is prepared as in Example 1, except the surfactant possesses a lipophilic component comprised of collagen, the hydrophilic component is a polyethylene glycol, the chelating agent is a carbonate, the ceramic is a carbonate apatite, and the growth factor bone morphogenic protein is distributed throughout the ceramic nanoparticle.

### Example 10

In this Example, a powder is prepared as in Example 1, except the surfactant possesses a lipophilic component comprised of an integrin, the hydrophilic component is a polyethylene glycol, the chelating agent is a carbonate, and the ceramic is a carbonate apatite.

### Example 11

In this Example, a powder is prepared as in Example 1, except the surfactant possesses a lipophilic component comprised of an IgG1 antibody, the hydrophilic component is polyethylene imine, the chelating agent is ethylene diamine triacetic acid, the ceramic is tricalcium phosphate, and the chemotherapeutic agent paclitaxel is distributed throughout the ceramic nanoparticle.

We claim:

1. A method of forming a surface functionalized nanoparticle comprising:
i) synthesizing a surfactant comprising a lipophilic component A, a hydrophilic component B, and a chelating agent X,
ii) forming a microemulsion using the surfactant,
iii) adding ceramic precursors to the microemulsion,
iv) precipitating ceramic nanoparticles within the microemulsion, and
v) attaching of the surfactant to the ceramic nanoparticle surface.

2. The method of claim 1, wherein the lipophilic component A is selected from a group comprising a polyester, a carbohydrate, and a peptide.

3. The method of claim 1, wherein the hydrophilic component B is selected from a group comprising a polyether, a polyamine, and an organophosphate.

4. The method of claim 1, wherein the chelating agent X is selected from a group comprising a carbonate, a carboxylate, a phosphate, a polyether, and a polyamine.

5. The method of claim 1, wherein the ceramic precursor is selected from the group comprising calcium chloride, calcium nitrate, sodium phosphate, ammonium phosphate and phosphoric acid.

6. The method of claim 1, wherein the surface functionalized nanoparticle further comprises at least one therapeutic agent.

7. The method of claim 1 further comprising isolating and purifying the surface functionalized nanoparticle wherein purifying further comprises precipitation, hydrothermal curing and/or grinding the nanoparticle.

8. The method of claim 1 wherein the surfactant comprises a biodegradable polymer.

9. A surface functionalized ceramic nanoparticle comprising a formula
(S_{ABX})—C;
wherein S describes a surfactant comprising components of A, B, and X; and C;
A is a lipophilic component;
B is a hydrophilic component;
X is a chelating agent; and
C is a ceramic forming component.

10. The nanoparticle of claim 9 wherein the lipophilic component A is selected from a group comprising a polyester, a carbohydrate, and a peptide.

11. The nanoparticle of claim 9 wherein the hydrophilic component B is selected from a group comprising a polyether, a polyamine, and an organophosphate.

12. The nanoparticle of claim 9 wherein the chelating agent X is selected from a group comprising a carbonate, a carboxylate, a phosphate, a polyether, and a polyamine.

13. The nanoparticle of claim 9 wherein the ceramic precursor is selected from the group comprising calcium chloride, calcium nitrate, sodium phosphate, ammonium phosphate and phosphoric acid.

14. The nanoparticle of claim 9 further comprising a therapeutic agent.

15. The nanoparticle of claim 9 wherein the shell comprises a biodegradable polymer.

16. An article comprising a surface functionalized ceramic nanoparticle comprising a formula:
(S_{ABX})—C;
wherein S describes a surfactant comprising components of A, B, and X; and C;
A is a lipophilic component;
B is a hydrophilic component;
X is a chelating agent; and
C is a ceramic forming component.

17. The article of claim 16 comprising a medical device selected from the group comprising an orthopedic reinforcing member, a bone cement, and a drug delivery device.

18. The article of claim 17 comprising a biodegradable fiber, fabric, tube, film, sheet, container or a molded part.

19. The article of claim 16 comprising a clarified biodegradable plastic.

20. The article of claim 16 comprising a therapeutic agent.
